(19) United States
(12) Patent Application Publication
(10) Pub. No.: US 2026/0248711 A1
(43) Pub. Date: Aug. 27, 2026

(54) AQUEOUS COMPOSITIONS COMPRISING A WATER-SOLUBLE POLYMER SYSTEM AND POLYOL

(71) Applicant: L'OREAL, PARIS (FR)

(72) Inventors: Monika LUSHKA, TOWACO, NJ (US); Donald KRONENBERG, AMITYVILLE, NY (US)

(73) Assignee: L'OREAL, PARIS (FR)

(21) Appl. No.: 19/059,878

(22) Filed: Feb. 21, 2025

(51) Int. Cl. A61K 8/81; A61K 8/34; A61K 8/73; A61Q 1/00

(52) U.S. Cl. CPC A61K 8/8182; A61K 8/345; A61K 8/737; A61K 8/8152; A61Q 1/00; A61K 2800/30; A61K 2800/5422; A61K 2800/594; A61K 2800/805

(57) ABSTRACT

Compositions useful for priming the skin prior to applying a color cosmetic composition thereto. The compositions include water, polyol, non-ionic surfactant and a water-soluble polymer system. The water-soluble polymer system includes vinyl pyrrolidone polymer; and crosslinked, unsulfonated acrylic polymer; The compositions are substantially free of fatty compounds, colorants and C2-C5 monoalcohols. Methods are also provided.

### FIELD OF THE INVENTION

The present invention relates to cosmetic primer compositions including a water-soluble polymer system and polyol.

### DISCUSSION OF THE BACKGROUND

A person's makeup routine may include combining various products to achieve a desired result, but the process often involves making compromises on various desirable attributes. For example, a primer might provide desirable transparency on the skin, but may not feel lightweight and/or have a desirably amount of tack or “hold.”

The present inventors have found that compositions having a particular polymer system, polyol and nonionic surfactant have an unexpectedly desirable combination of properties, particularly for priming the skin before applying color cosmetics.

### SUMMARY OF THE INVENTION

The present invention relates to compositions useful as cosmetic primers. The compositions include water, a water-soluble polymer system, polyol, and non-ionic surfactant. The compositions are further substantially free of fatty compounds, colorants and C2-C5 monoalcohols. The water-soluble polymer system includes vinyl pyrrolidone polymer, and crosslinked, unsulfonated acrylic polymer and optionally arabinogalactan gum.

According to certain notable embodiments, the compositions include about 50% to about 80% of water; about 15% to about 40% polyol; about 1.25% to about 3% non-ionic surfactant and about 2% to about 4% of a water-soluble polymer system, where all concentrations are by weight. The water-soluble polymer system includes vinyl pyrrolidone polymer; crosslinked, unsulfonated acrylic polymer; and optionally arabinogalactan gum. The compositions are substantially free of fatty compounds, colorants and C2-C5 monoalcohols.

According to other embodiments, the compositions are also substantially free of sulfonated acrylic polymers and/or substantially free of organic UV filters such as oil-soluble organic UV filters.

According to certain other embodiments, the combined concentration of the water and the polyol is present in a concentration by weight of at least about 80%.

According to other embodiments, water is present in a concentration by weight of from about 55% to about 75%, wherein the polyol is present in a concentration by weight of from about 15% to about 35%, and the water-soluble polymer system is present in a concentration by weight from about 2% by weight to about 3%.

According to other embodiments the crosslinked, unsulfonated acrylic polymer is present in a concentration by weight of from about 0.5% to about 1.0%.

According to other embodiments the non-ionic surfactant includes a high HLB sorbitan ester surfactant.

According to certain other embodiments, the composition includes a plurality of or even all of the limitations specified above in this and the preceding two paragraphs.

According to another aspect of the invention, a method of making up the skin, comprising applying a priming composition to said skin prior to applying a color cosmetic composition thereto, wherein the priming composition includes water, a water-soluble polymer system and polyol. The compositions are further substantially free of fatty compounds, waxes, colorants and C2-C5 monoalcohols. The water-soluble polymer system includes vinyl pyrrolidone polymer, and crosslinked, unsulfonated acrylic polymer, and optionally arabinogalactan gum.

It is to be understood that both the foregoing general description and the following detailed description are exemplary and explanatory only and are not restrictive of the invention.

### DETAILED DESCRIPTION OF THE INVENTION

As used herein, the expression, “a” or “an” as used herein means “at least one.” “At least one” means one or more and thus includes individual components as well as mixtures/combinations.

As used herein, all ranges provided are meant to include every specific range within, and combination of sub ranges between, the given ranges. Furthermore, notably the range description of the type “from about 1%, 2% or 3% to about 5%, 10% or 15%,” includes about 1% to about 5%, about 1% to about 10%, about 1% to about 15%, about 2% to about 5%, about 2% to about 10%, about 2% to about 15%, about 3% to about 5%, about 3% to about 10%, and/or about 3% to about 15%. Other than in the operating examples, or where otherwise indicated, all numbers expressing quantities of ingredients and/or reaction conditions are to be understood as being modified in all instances by the term “about,” meaning within 10% to 15% of the indicated number. So “about 10%” means from 8.5% to 11.5%, such as from 9% to 11%.

“Active basis” as used herein means considering only the particular component of ingredient (e.g., in a composition) and ignoring other chemically unrelated components that may also be present in the same raw material source of that particular component.

“Polymer” as used herein means a compound which is made up of at least two monomers.

“Keratinous materials” includes materials containing keratin such as hair, skin, eyebrows, lips and nails and in particular, skin, such as facial skin.

“Solids basis” as used herein means considering only components (e.g., in a composition) that are solid at room temperature and ignoring portions of the composition that are liquid, e.g., water and other volatile solvents.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, hydroxyalkyl groups, and polysiloxane groups. The substituent(s) may be further substituted.

“Volatile,” as used herein, means having a flash point of less than about 100° C.

“Non-volatile,” as used herein, means having a flash point of greater than about 100° C.

“Substantially free” means having less than 1% by weight of a particular component, preferably less than 0.5% by weight, such as less than 0.25% by weight, such as less than 0.1% by weight, such a less than about 0.05% by weight.

“Anhydrous” means the compositions are substantially free of water, and preferably have no water.

All percentages of ingredients herein are listed on an actives basis and on a weight basis with respect to the entire composition unless specifically stated otherwise.

The composition of the present invention may be of various forms including semi-solid, and fluid under shear.

The composition of the invention generally include (1) water, (2) a water-soluble polymer system, and (3) polyol. These components may co-exist in a (e.g. single or sole) aqueous phase of the composition. Various other optional ingredients such as preservatives, skin active ingredients, emulsifiers, and the like may also be dissolved or dispersed in the aqueous phase.

The compositions and methods of the present invention can comprise, consist of, or consist essentially of the essential elements and limitations of the invention described herein, as well as any additional or optional ingredients, components, or limitations described herein or otherwise useful in personal care.

The present disclosure is drawn to a facial makeup product, such as a primer, that provides a lightweight, fresh sensation while also providing enhancing the wear of makeup applied under or over the product while also remaining transparent (e.g., no white cast) on the skin and providing an appropriate amount of tack or hold.

The disclosed makeup composition may be in the form of a gel and hence may be a “gel composition.”

“Gel composition” means a composition which does not flow similar to a liquid when applied to a substrate, a composition which has a 3-dimensional network that inhibits the composition from spreading on, or dripping from, a substrate after application owing to gravity over a short period of time (e.g., less than 10 seconds). G′ (storage modulus) is higher than G″ (loss modulus) at all range of strains up to 300% strain. Preferably, G′ (storage modulus) is higher than G″ (loss modulus) at low strain, but with G′ decreasing and G″ increasing, and the gel has the crossover point at >0.1% strain, preferably >1% strain, and preferably less than 200% strain, preferably less than 150% strain.

“Gel Crossover Point” (Sol/Gel Point), means the point at which the G″ (loss modulus) intersects the G′ (storage modulus), reported in % strain. It is the point at which a composition goes from a more solid state to a more liquid state. An example of a method for determining gel crossover point is as follows: G″ (loss modulus) and G′ (storage modulus) using a Discovery HR-3 Rheometer by TA Instruments, having 40 mm parallel plate geometry on a stainless steel flat peltier plate. The test can be run @25° C., with test parameter of angular frequency of 1.0 rad/s and logarithmic sweep: Strain % 0.01 to 1000.0%. 5 points per decade. Results reported in % strain.

### Water

Compositions described herein include water. The water may be present in a concentration by weight ranging from about 30% to about 95%, such as from about any of 40%, 50%, or 55% by weight to about any of 55%, 60%, 65%, 75%, 80%, 85% or 95% by weight with respect to the total weight of the composition.

### Water-Soluble Polymer System

Compositions disclosed herein include a water-soluble polymer system. The water-soluble polymer system includes at least one water-soluble polymer. The term “a water-soluble polymer” may leave a film (e.g., a continuous film) on the substrate to which it is applied.

The total amount of water-soluble polymers may be present in a concentration by weight ranging from about 0.1%, 0.5%, 1.0%, 1.25%, 1.5%, 2.0%, or 2.5% to 2.5%, 3.0%, 4.0%. 4.5%, or 5% by weight with respect to the total weight of the composition.

The water-soluble polymer system described herein may include a vinyl pyrrolidone polymer; and a crosslinked, unsulfonated acrylic polymer. It may further optionally include arabinogalactan gum. In other embodiments, the water-soluble polymer system includes all of an arabinogalactan gum, a vinyl pyrrolidone polymer; and a crosslinked, unsulfonated acrylic polymer.

### Vinyl Pyrrolidone Polymer

Compositions disclosed herein include a vinyl pyrrolidone polymer. According to certain embodiments the vinyl pyrrolidone polymer is or includes a vinylpyrrolidone homopolymer. Vinylpyrrolidone homopolymers (also known as polyvinylpyrrolidone) are water-soluble polymers chemically composed of repeating 5-membered heterocyclic rings i.e., C₆H₉NO units. Vinylpyrrolidone homopolymers useful in the present invention are high molecular weight. By “high molecular weight” it is meant having a weight average molecular weight (M_{w}) that is at least about 200,000 daltons. In certain embodiments of the invention the M_{w} of the vinylpyrrolidone homopolymer is at least about 350,000 daltons. According to certain other embodiments of the invention, in order to enhance self-curling and assist in the ease of application to lashes, the vinylpyrrolidone homopolymer has an M_{w} that is from about 200,000 daltons to about 3,000,000 daltons such as from about 500,000 daltons to about 1,500,000 daltons.

In certain embodiments, the high molecular weight vinylpyrrolidone homopolymer may be a commercially available variety, such as PVP K-60 (or Plasdone® K-60), PVPK-90 (or Plasdone® K-90), or PVP K-120 (or Plasdone® K-120), each commercially available from Ashland, Inc of Kovington, Ky.

The vinyl pyrrolidone polymer may be present in a concentration by weight ranging from about 0.5% or 0.75, or 1% to about 1%, 2%, or 3% by weight with respect to the total weight of the composition.

### Crosslinked, Unsulfonated Acrylic Polymer

In accordance with the present invention, compositions may include at least one crosslinked, unsulfonated acrylic polymer.

The crosslinked, unsulfonated acrylic polymer is formed from at least one ethylenically unsaturated monomer, each of which is free of sulfonated moieties.

Preferably, the crosslinked, unsulfonated acrylic polymer is an acrylic emulsion polymer (formed from emulsion polymerization). According to certain embodiments, the crosslinked, unsulfonated acrylic polymer is an alkali-swellable acrylic emulsion polymer. The performance of this polymer in the composition may be from neutralization of the protonated carboxyl groups which then ionize, thereby expanding the polymer due to charge repulsion. In certain embodiments, the acrylic thickening polymer free of sulfonated monomers is further hydrophobically modified, such as with one or more alkyl groups.

Suitable acrylic thickening polymer free of sulfonated monomers include CARBOPOL polymers such as CARBOPOL 2984, CARBOPOL 5984, CARBOPOL 940, CARBOPOL 941, CARBOPOL 980, CARBOPOL 981, CARBOPOL Clear Polymer, CARBOPOL SC-200, CARBOPOL SC-800, CARBOPOL Silk-100, CARBOPOL Ultrez-10, CARBOPOL Ultrez-20, CARBOPOL Aqua SF-1. CARBOPOL polymer are available from Lubrizol.

According to preferred embodiments the acrylic thickening polymer free of sulfonated monomers is a hydrophobically-modified, crosslinked acrylic polymer such as CARBOPOL Ultrez-20.

The acrylic thickening polymer free of sulfonated monomers may be present in a concentration by weight ranging from about 0.5% or 0.75, or 1% to about 1%, 1.25%, 1.5%, or 2% by weight with respect to the total weight of the composition.

### Arabinogalactan Gum

The arabinogalactan gum generally includes a polysaccharide component that is a heteropolysaccharide and/or a salt thereof. The polysaccharide component generally includes arabinose and galactose residues. According to certain embodiments, these include a backbone of galactose and side chains that include arabinose and optionally other saccharides such as rhamnose and glucaronic acid. Arabinose is a pentose sugar having a 5 carbon atom structure. In addition to the polysaccharide component, the gum may further include a glycoprotein component that includes one or more glycoproteins.

According to certain notable embodiments, the arabinogalactan gum is gum Arabic. Gum Arabic is a dried exudate from the trunks and branches of several species of African acacia (Acacia senegal (L.) Willd. and related species) such as Acacia senegal or Vachellia (Acacia) seyal in the family Fabaceae (Leguminosae).

Gum Arabic is also known as acacia gum and may be also known under names such as Kordofan Gum, Gum Senegal, Acacia Vera, Gummi Africanum, Gummae Mimosae, kher, Sudan Gum Arabic, Somali Gum, Yellow Thorn, Mogadore Gum, Indian Gum and Australian Gum, Gum sudani. These may be harvested commercially from wild trees, mostly in Sudan and throughout from Senegal to Somalia.

The arabinogalactan gum may be present in a concentration by weight ranging from about 0.05% or 0.1% to about 0.1%, 0.2%, 0.25%, 0.3%, 1% or 2% by weight with respect to the total weight of the composition.

According to certain embodiments vinyl pyrrolidone polymer; the crosslinked, unsulfonated acrylic polymer; and the optional arabinogalactan gum collectively comprise 90% percent or more by weight of the water-soluble polymer system. According to certain other embodiments, the high molecular weight vinylpyrrolidone homopolymer comprises 99% or more, such as 100% of the water-soluble polymer system.

The water-soluble polymer system may include one or more additional water-soluble polymers, such as those that are commonly used in makeup compositions. According to certain embodiments compositions disclosed herein are substantially free of sulfonated acrylic polymers such as those including or formed from 2-Acrylamido-2-methylpropane sulfonic acid (AMPS polymers).

### Polyol

Compositions of the present invention further include polyol, such as polyols that are fully miscible with water, such as throughout all weight ratios with water, and/or such as polyols that are free of uninterrupted carbon chains (—C—) of more than 7 such as more than 8, such as more than 9 carbon atoms.

The polyol may have from 2 to 10 carbon atoms, preferably from 2 to 6 carbon atoms. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

The polyols are preferably di- to octa-hydric polyols. Examples of the di- to octa-hydric polyols include dihydric alcohols, such as propylene glycol, butylene glycol, hexylene glycol, hexamethylene glycol, and neopenthyl glycol; trihydric alcohols, such as glycerol, trioxy isobutane, 1,2,3-butanetriol, 1,2,3-pentanetriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4,5-heptanetriol, 2,4-dimethyl-2,3,4-pentanetriol, pentamethylglycerol, pentaglycerol, 1,2,4-butanetriol, 1,2,4-pentanetriol, trimethylolethane, and trimethylolpropane; tetrahydric alcohols, such as pentaerythritol, 1,2,3,4-pentanetetrol, 2,3,4,5-hexanetetrol, 1,2,4,5-pentanetetrol, and 1,3,4,5-hexanetetrol; pentahydric alcohols, such as adonitol, arabitol, and xylitol; hexahydric alcohols, such as dipentaerythritol, sorbitol, mannitol, and iditol; and octahydric alcohols, such as sucrose. Particularly notable polyols include propylene glycol, hexylene glycol and glycerol.

In certain embodiments, the polyol is unable to depress the surface tension of water to less than 50 mN/m at 0.1% in deionized water.

The polyol may be present in a concentration by weight ranging from about 10%, 15%, or 20% to about 20%, 30%, 35% or 40% by weight with respect to the total weight of the composition.

According to certain embodiments the combined concentration (sum of their concentrations) of the water and the polyol is at least about 80%.

### C2-C5 Monoalcohols

In order to avoid introducing too much initial tackiness sensation, the compositions described herein, as noted previously, may be substantially free of C2-C5 monoalcohols. Examples of C2-C5 monoalcohols include ethanol, propanol, butanol, pentanol, isopropanol, isobutanol and isopentanol. Ethanol is particularly notable.

In order to avoid introducing undesirable coloration or opacification, compositions described herein, as noted previously, may be substantially free of colorants. Examples of colorants include inorganic pigments such as one or more color pigments and, in certain other embodiments also opacifying pigments such as titanium dioxide.

Color pigments generally provide hiding powder to aid in concealing skin imperfections while imparting some additional visible color. Color pigments include any of various inorganic pigments such as iron oxides, ultramarine blue pigments, manganese violet, ferric ferrocyanide and chromium green pigments, carbon black, and the like. Iron oxide is particularly notable. Color pigments may be coated or uncoated. Opacifying pigments such as titanium dioxide provide hiding powder without introducing color.

### Fatty Compounds

As described above, compositions of the present invention are substantially free of fatty compounds. By “fatty compounds,” it is meant any of various fatty substances insoluble in water and including, for example, hydrocarbon based fatty substances or silicone-based fatty substances. These may be liquid or solid at room temperature and may be volatile or non-volatile. In certain embodiments, the fatty compounds are unable to depress the surface tension of water to less than 50 mN/m at 0.1% in deionized water.

Examples of fatty compounds include oils. By “oil,” it is meant compounds having a melting point of less than about 15° C., such as less than about 10° C., and generally insoluble in water and includes a hydrophobic moiety. The oil may meet one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the oil does not include hydroxyl moieties. The oil may have a weight average molecular weight less than about 1000 daltons.

Examples of oils include vegetable oils (glyceryl esters of fatty acids, monoglycerides, diglycerides, triglycerides) and fatty esters. Specific non-limiting examples include, without limitation, esters such as isopropyl palmitate, isopropyl myristate, isononyl isonanoate C12-C15 alkyl benzoates, caprylic/capric triglycerides, ethylhexyl palmitate, silicone oils (such as dimethicone and cyclopentasiloxane), pentaerythritol tetraoctanoate and mineral oil. Other examples of oils include liquid organic ultraviolet filter commonly used for example as UV-absorbing sunscreens such as octocrylene, octyl salicylate, octyl methoxyxcinnamate, among others.

Examples of oils include volatile and/or non-volatile oils. Such oils can be any acceptable oil including but not limited to silicone oils and/or hydrocarbon oils.

Examples of volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C.

Other fatty compounds that the compositions disclosed herein may be substantially free of include waxes. As used herein, “wax” is intended to mean a lipophilic fatty compound that is solid at room temperature (about 25° C.) and atmospheric pressure (760 mm Hg, i.e., 105 Pa), which undergoes a reversible solid/liquid change of state and which has a melting point of greater than 30° C., and in some embodiments, greater than about 55° C. up to about 120° C. or even as high as about 200° C. In certain embodiments the wax has a weight average molecular weight of less than about 2000 daltons.

Waxes may change from the solid to the liquid state reversibly, and/or may have a hardness of more than 0.5 MPa at ambient temperature, and an anisotropic crystalline organization in the solid state.

The term wax may include waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candelilla wax, ouricurry wax, cork fiber waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes (linear, low molecular weight polyethylene waxes), waxes obtained by Fischer-Tropsch synthesis, waxy copolymers and their esters, and silicone and fluoro waxes.

The term wax may further include high melting point hydrogenated oils of animal or plant origin. Examples include hydrogenated jojoba waxes and hydrogenated oils which are obtained by catalytic hydrogenation of fats composed of a C8-C32 linear or nonlinear fatty chain, hydrogenated sunflower oil, hydrogenated castor oil, hydrogenated copra oil, hydrogenated lanolin and hydrogenated palm oils.

In certain embodiments of the invention, the fatty compounds that compositions disclosed herein are substantially free of may not fall into the definition of wax or oils, such as fatty materials that are semi-solid, pastes or paste-like at room temperature (“paste-like compound”), e.g., have a melting temperature in a range from about 15° C. to about 40° C. Examples include shea butter, hydrogenated butters, and any of various partial esters of diglycerin with medium chain fatty acids, such as Bis-Diglyceryl Polyacyladipate-2, commercially available as SOFTISAN 649 from Cremer Oleo of Hamburg, Germany; and ethylhexyl hydroxystearate such as WICKENOL 171 available from Alzo International of Sayerville, NJ.

### Nonionic Surfactant

As mentioned above, compositions useful in the present invention include a nonionic surfactant. Examples of nonionic surfactants that may be used include, for example, nonionic surfactants such as those having a high HLB.

According to certain embodiments of the present invention, compositions may include at least one high HLB surfactant. “HLB” refers to the “hydrophilic-lipophilic balance” associated with emulsifiers/surfactants. In particular, “HLB” value relates to the ratio of hydrophilic groups and lipophilic groups in emulsifiers, and also relates to solubility of the emulsifiers. Lower HLB emulsifiers are more soluble in oils (lipophilic material) and are more appropriate for use in water-in-oil (W/O) emulsions. Higher HLB emulsifiers are more soluble in water (hydrophilic material) and are more appropriate for oil-in-water (O/W) emulsions. In the context of the present invention, “high HLB surfactant” means a surfactant having an HLB value of greater than 8. Also, in the context of the present invention, “high HLB value” means a surfactant having an HLB value greater than or equal to 8, preferably greater than or equal to 9, preferably greater than or equal to 10, preferably greater than or equal to 12, and preferably greater than or equal to 13.

Examples of surfactants include, but are not limited to, the following:
Polyethylene glycolated (PEG) and/or glycerolated esters or ethers, such as polyethylene glycolated and/or glycerolated branched or linear C8-C24 compounds, for example, Oleth-50, Oleth-20, Oleth-10, Ceteth-10, Steareth-20, Laureth-23, PEG-8 stearate, PEG-8 isostearate, PEG-20 stearate PEG-40 stearate, PEG-40 hydrogenated castor oil, etc.;
sorbitan esters or ethers, such as oxyethylenated or nonoxyethylenated sorbitan mono- or polyalkyl esters or ethers, such as high HLB sorbitan esters. Examples of oxyethylenated sorbitan mono- or polyalkyl esters include Polysorbate 21, Polysorbate 40, Polysorbate 80, Polysorbate 60, Polysorbate 61. Examples of nonoxyethylenated sorbitan mono- or polyalkyl esters include sorbitan isostearate, glyceryl sorbitan isostearate, sorbitan sesquioleate, sorbitan laurate, sorbitan monopalmitate, sorbitan oleate, sorbitan trioleate, sorbitan monostearate and sorbitan tristearate. Polysorbate-80 is a notable high HLB oxyethylenated sorbitan mono- or polyalkyl ester.

Sugar mono- or polyalkyl esters or ethers, such as the mono- or polyalkyl esters or ethers of sugars as described in the patent U.S. Pat. No. 6,689,371. Mention may be made, for example, of methyl glucose isostearate, sucrose distearate, sucrose stearate, etc.

The concentration by weight of nonionic surfactant, may be from about 1%, 1.5% or 2%, such as from about 2%, 2.5%, 3%, 4% or 5%.

### Other Functional Ingredients

Other functional ingredients that may be included in compositions disclosed herein include: fragrances; water-soluble sunscreens, skin active agents, and emulsifiers. A person skilled in the art will take care to select the optional additional ingredients and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

According to certain embodiments compositions disclosed herein are substantially free of organic ultraviolet (UV) filters such as those that are water-insoluble and commonly used in recreational sunscreen products or other “SPF” products. Examples include avobenzone, oxybenzone, homosalate, among others known by those of ordinary skill in the art.

The other functional ingredients may be present in the composition in concentrations up to about 20%, such as less than about 15%, such as less than about 10%, such as from about 0%, 2%, or 5% to about 5%, 10%, 15%, or 20%, including all ranges and subranges therebetween.

According to certain embodiments the compositions include about 55% to about 75% by weight of water; about 2% to about 4% of a water-soluble polymer system comprising vinyl pyrrolidone polymer; and crosslinked, unsulfonated acrylic polymer; about 55% to about 75% by weight of water; about 2% to about 4% of a water-soluble polymer system that includes vinyl pyrrolidone polymer and crosslinked, unsulfonated acrylic polymer. The composition further includes about 15% to about 40% polyol; and about 1.25% to about 3% non-ionic surfactant, and wherein the composition is substantially free of fatty compounds, colorants and C2-C5 monoalcohols.

In certain other embodiments, the compositions disclosed herein are substantially free of esters that are not surfactants (non-surfactant esters), such as non-surfactant esters having water solubility (e.g., Polyglyceryl-10 Eiconsanedioate/Tetradecanedioate).

According to preferred embodiments of the present invention, methods of treating, caring for and/or making up a keratinous material by applying compositions of the present invention to the keratinous material in an amount sufficient to treat, care for and/or make up the keratinous material are provided. According to yet other preferred embodiments, methods of enhancing the appearance of a keratinous material by applying compositions of the present invention to the keratinous material in an amount sufficient to enhance the appearance of the keratinous material are provided.

In accordance with the preceding preferred embodiments, the compositions of the present invention are applied topically to the desired area of the keratinous material in an amount sufficient to treat, care for and/or make up the keratinous material, to prime the skin prior to treatment with a color cosmetic.

Accordingly, methods of the present invention may include applying a priming composition to said skin prior to applying a color cosmetic composition thereto, wherein the priming composition includes water; a water-soluble polymer system such as one including arabinogalactan gum, vinyl pyrrolidone polymer; and crosslinked, unsulfonated acrylic polymer; and polyol; wherein the composition is substantially free of fatty compounds and C2-C5 monoalcohols.

The compositions may be applied to the desired area as needed, preferably once daily, and then preferably allowed to dry before subjecting to contact such as with clothing or other objects. Preferably, the composition is allowed to dry for about 4 minutes or less, more preferably for about 2 minutes or less.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Example 1—Compositions

The following compositions were prepared and evaluated for sensory attributes. Compositions consistent with the ingredients and concentrations above were prepared by combining the ingredients and mixing until homogeneous.

The concentrations of various ingredients in the compositions (active basis) and sensory results/comments are shown in Table 1.

TABLE 1
Test Compositions and Sensory Results

| | | | | | Gum | Non-ionic | | Result and |
|---|---|---|---|---|---|---|---|---|
| | Water * | Polyol | PVP | Carbomer | Acacia | Surfactant | Other * | Comments |
| E1 | 69 | 20 | — | 0.8 | 0.1 | 1.5 | 10 | Poor film |
| | | | | | | | | quality and |
| | | | | | | | | poor makeup |
| | | | | | | | | wear. |
| E2 | 68 | 20 | 2.0 | — | 0.1 | 1.5 | 10 | Unacceptably |
| | | | | | | | | thin. |
| E3 | 67 | 16 | 2.0 | 1.2 | 0.6 | 0 | 13 | Difficult to |
| | | | | | | | | spread/ |
| | | | | | | | | draggy/ |
| | | | | | | | | patchy |
| | | | | | | | | application |
| E4 | 57 | 31 | 2.0 | 0.9 | — | 1.15 | 9 | Thick; |
| | | | | | | | | difficult to |
| | | | | | | | | spread/ |
| | | | | | | | | draggy/ |
| | | | | | | | | patchy |
| | | | | | | | | application |
| E5 | 61 | 31 | 2.0 | 0.6 | — | 1.5 | 5 | Good |
| | | | | | | | | performance. |
| E6 | 69 | 20 | 2.0 | 0.6 | 3 | 1.5 | 5 | Too tacky. |
| E7 | 67 | 20 | 2.0 | 0.8 | 0.1 | 1.5 | 10 | Good |
| | | | | | | | | performance. |
| E8 | 67 | 20 | 2.0 | 0.8 | — | 1.5 | 10 | Good |
| | | | | | | | | performance. |
| E9 | 65 | 19 | 2.0 | 0.8 | — | 1.5 | 10 ** | Increased |
| | | | | | | | | tack vs E7 |
| E10*** | N/A | N/A | N/A | N/A | N/A | N/A | N/A | Sticky |
| (E.L.F.) | | | | | | | | |

* The other ingredients include preservatives, and skin active ingredients Other ingredients in the compositions (E1-E9) include 5% by weight of fruit water (except E6 which had only 1% fruit water). The fruit water contributes additional water to the composition.
** E9 included 5% ethanol in the composition and the ethanol is included amoung the other ingredients
***E10 is ELF Power Grip Primer available from E.L.F. Cosmetics of Oakland, California and includes PEG-240/Hdi Copolymer Bis-Decyltetradeceth-20 Ether, not the polymer system disclosed herein

The various compositions were evaluated for behavior (thickness/thinness), spreadability on the skin, perception of tack, stickiness, and, effect on subsequent color cosmetic (foundation) application and wear.

It can be seen from the results that both vinyl pyrrolidone polymer (e.g., PVP) and crosslinked, unsulfonated acrylic polymer (e.g., carbomer) were needed in the composition to produce proper viscosity and film quality/makeup result.

Additionally, tack for select compositions were quantitatively evaluated using a Texture Analyzer (TA Instruments, New Castle, Delaware) using a P/0.5 HS probe with a 0.5 ml film spread on a steel plate. Peak negative force was measured after 30 seconds. The data is reported below in Table 2:

TABLE 2

| Test Composition | Tack at 30 seconds (g) |
|---|---|
| E10 | 4.1 |
| E7 | 3.1 |
| E9 | 4.5 |

It can also be seen that adding alcohol to E7 (see E9), tack at 30 seconds (which can be undesirable to consumers) rose to levels similar to E10.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. accordingly, the description is not intended to limit the invention to the form disclosed herein. Also, it is intended to the appended claims to construed to include alternative embodiments.

What is claimed is:

1. A composition, comprising:
about 50% to about 80% by weight of water;
about 2% to about 4% of a water-soluble polymer system comprising:
vinyl pyrrolidone polymer; and
crosslinked, unsulfonated acrylic polymer;
about 15% to about 40% polyol; and
about 1.25% to about 3% non-ionic surfactant, and wherein the composition is substantially free of fatty compounds, colorants and C2-C5 monoalcohols.

2. The composition of claim 1 wherein the composition is substantially free of sulfonated acrylic polymers.

3. The composition of claim 1 wherein the composition is substantially free of organic UV filters.

4. The composition of claim 1 wherein the water-soluble polymer system further comprises arabinogalactan gum.

5. The composition of claim 1 wherein the combined concentration of the water and the polyol is present in a concentration by weight of at least about 80%.

6. The composition of claim 1 wherein water is present in a concentration by weight of from about 55% to about 75%, wherein the polyol is present in a concentration by weight of from about 15% to about 35%, and wherein the water-soluble polymer system is present in a concentration by weight from about 2% by weight to about 3% by weight.

7. The composition of claim 1 wherein the crosslinked, unsulfonated acrylic polymer is present in a concentration by weight of from about 0.5% to about 1.0%.

8. The composition of claim 1 wherein the non-ionic surfactant includes a high HLB sorbitan ester surfactant.

9. A method of making up the skin, comprising applying a priming composition to said skin prior to applying a color cosmetic composition thereto, wherein the priming composition comprises:
about 50% to about 80% by weight of water;
about 2% to about 4% of a water-soluble polymer system comprising:
vinyl pyrrolidone polymer; and
crosslinked, unsulfonated acrylic polymer;
about 15% to about 40% polyol; and
about 1.25% to about 3% non-ionic surfactant, and wherein the composition is substantially free of fatty compounds, colorants and C2-C5 monoalcohols.

10. The method of claim 9, wherein the composition is substantially free of sulfonated acrylic polymers.

11. The method of claim 9, wherein the composition is substantially free of organic UV filters.

12. The method of claim 9, wherein the water-soluble polymer system further comprises arabinogalactan gum.

13. The method of claim 9, wherein the combined concentration of the water and the polyol is present in a concentration by weight of at least about 80%.

14. The method of claim 9, wherein the water is present in a concentration by weight of from about 55% to about 75%, wherein the polyol is present in a concentration by weight of from about 15% to about 35%, and wherein the water-soluble polymer system is present in a concentration by weight from about 2% by weight to about 3% by weight.
